(19) United States
(12) Patent Application Publication
(10) Pub. No.: US 2026/0207552 A1
(43) Pub. Date: Jul. 23, 2026

(54) MELATONIN LIQUID FORMULATIONS

(71) Applicant: ITF RESEARCH PHARMA, S.L.U., Alcobendas, Madrid (ES)

(72) Inventors: Pedro Enrique ESQUINAS GONZÁLEZ, Alcobendas, Madrid (ES); Patricia GARCIA RODRIGUEZ, Alcobendas, Madrid (ES); Katia URSO, Alcobendas, Madrid (ES)

(21) Appl. No.: 19/138,879

(22) PCT Filed: Dec. 15, 2023
(86) PCT No.: PCT/EP2023/086030

§ 371 Date: Jun. 13, 2025

(30) Foreign Application Priority Data
Dec. 16, 2022 (EP) 22383234.6

(51) Int. Cl. A61K 31/4045; A61K 9/00; A61K 9/10; A61K 47/14; A61K 47/36; A61K 47/44

(52) U.S. Cl. CPC A61K 31/4045; A61K 9/0053; A61K 9/10; A61K 47/14; A61K 47/36; A61K 47/44

(57) ABSTRACT

The present invention provides liquid formulations of melatonin based on an oily vehicle, methods for their preparation and their uses in medicine, more specifically in the treatment of sleep disorders such as insomnia and circadian rhythm sleep-wake disorders.

### FIELD OF INVENTION

The present invention relates to a liquid formulation, in particular to a suspension, of melatonin, methods for its preparation and its use in medicine, in particular for the treatment of sleep disorders such as insomnia and circadian rhythm sleep-wake disorders.

### BACKGROUND OF THE INVENTION

Melatonin is a hormone primarily released by the pineal gland at night. In vertebrates, it is involved in synchronizing circadian rhythms, including sleep-wake timing and blood pressure regulation, and in control of seasonal rhythmicity including reproduction, fattening, moulting and hibernation.

Melatonin is used as a dietary supplement or medication in the treatment of sleep disorders such as insomnia and circadian rhythm sleep-wake disorders.

Melatonin is approved as a medicine in the European Union for use in the short-term treatment of different types of insomnia and jet-lag, and it is administered in immediate- and controlled-release tablet forms.

Melatonin is also available as over-the-counter dietary supplements for sleep disturbances in many countries. It may be administered orally (as capsules, tablets, gummies or liquids), sublingually (as spray or tablets) or as transdermal patches.

Even though melatonin is widely used in oral solid forms, liquid formulations are demanded for children, particularly those under 3 years old (infants and toddlers), given that this segment of patients usually presents difficulties in swallowing medicines or dietary supplements in solid form, and liquid forms facilitate the ingestion.

A liquid formulation of melatonin should be not only technically feasible, but also physico-chemically stable, microbiologically acceptable, and ideally should have good organoleptic properties.

Melatonin is commercially available as oral liquid formulations, typically based on solely or predominantly aqueous vehicles. However, due to stability problems and/or poor organoleptic properties, these liquid forms usually contain preservatives (for instance: ethyl alcohol, sodium benzoate, potassium sorbate), viscosity enhancers (glycerine) and/or other type of excipients (sweeteners). Furthermore, such excipients are generally not suitable for administration to children under three years old.

Therefore, there is still a need in the art for formulations containing melatonin in a liquid form that demonstrate physicochemical and microbiological stability, good organoleptic properties and are acceptable for use in children under 3 years old, which offer additional advantages for medical practitioners as well as patients compared to the presently available formulations.

### SUMMARY OF THE INVENTION

The authors of the present invention have surprisingly found that a selection of an oily vehicle and a viscosity increasing agent, leads to oral liquid formulations, preferably suspensions, of melatonin that are physically, chemically and microbiologically stable under storage, in non-refrigerated conditions, for at least 6 months and have good palatability, thus resulting in improved patient and caregiver acceptance.

Specifically, the formulations show, at 6 months of storage, a loss of concentration of melatonin of at most 10%, preferably at most 7%, more preferably at most 5%, even more preferably at most 3%, yet more preferably at most 1%, with respect to the concentration of melatonin observed when the storage was initiated. More particularly, said storage can be at the condition 25° C./60% relative humidity (RH) and/or at 40° C./75% RH.

Besides, the formulations of the present invention keep their appearance and organoleptic properties during at least 6 months of storage at said conditions.

Additionally, the suspensions of the present invention show reduced sedimentation and can be easily reconstituted into a uniform suspension through slight agitation, if necessary, thus eliminating variations of melatonin concentration throughout the vehicle and ensuring the dose uniformity.

The inventors have further found that there is no need to add a preservative in order to prevent the microbial contamination of the liquid formulation of the present invention.

Moreover, they surprisingly found that it is not necessary to add any other excipient besides the oily vehicle and the viscosity increasing agent in order to obtain the physicochemically stable liquid formulation of melatonin of the present invention.

The present formulations can be used by patients of any age but are particularly suitable for use in pediatrics: they are in liquid form—which is the dosage form of choice for young children—, have a pleasant taste, contain the recommended starting dose of melatonin in this age group in a small volume (for instance, 4-6 drops) —which facilitates the administration, alone or mixed with foods and/or beverages-, and allow for an easy dose escalation according to the age of the patient.

Therefore, according to a first aspect, the invention is directed to a liquid formulation, preferably a suspension, comprising:
melatonin in particulate solid form,
a vehicle, which is an oil, preferably selected from medium chain triglycerides, vegetable oils and their mixtures, comprised in the liquid formulation in an amount of at least 80% w/v; and
a viscosity increasing agent, preferably selected from: silicon dioxide, such as colloidal silicon dioxide; a composition comprising at least 50% by weight of mono-, di- or tri-glycerides and mixtures thereof, such as a composition comprising glycerol monostearate or glyceryl dibehenate; a gum, such as xanthan gum; and their mixtures.

A second aspect of the present invention is directed to a method for preparing the liquid formulation of the first aspect of the invention, said process comprising the following steps:
a) Mixing the vehicle in liquid form with the viscosity increasing agent in particulate solid form;
b) Adding the melatonin in particulate solid form into the mixture obtained in step a) and mixing.

A further aspect of the present invention is directed to a liquid formulation according to the invention for use as a medicament and/or as a food supplement.

An additional aspect of the invention is directed to a liquid formulation according to the invention for use in the prevention or treatment of sleep disorders.

These aspects and preferred embodiments are additionally described further down in the description and defined in the claims.

### DESCRIPTION OF THE FIGURES

FIG. 1: Schematic overview of the manufacturing process of a formulation according to the present invention.

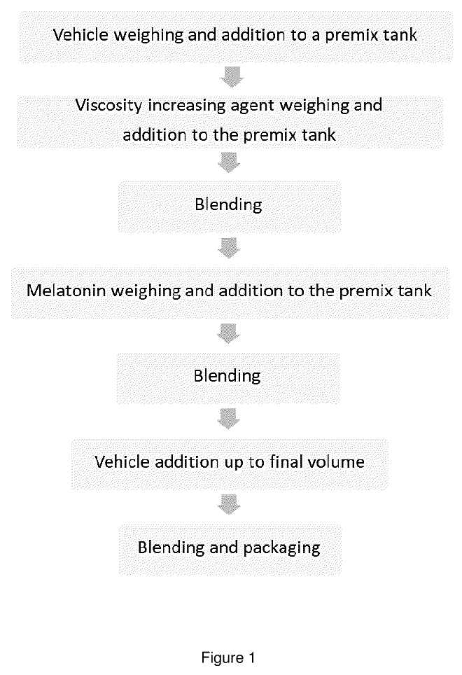

### DETAILED DESCRIPTION OF THE INVENTION

The present invention provides for a melatonin liquid formulation, methods for obtaining said formulation and uses thereof.

### Definitions

In order to facilitate the comprehension of the present invention, the meaning of some terms and expressions as used in the context of the invention are set forth below.

As used herein, melatonin (also known as N-[2-(5-methoxy-1H-indol-3-yl)ethyl] acetamide (CAS 73-31-4), N-acetyl-5-methoxytryptamine) is a compound of formula:

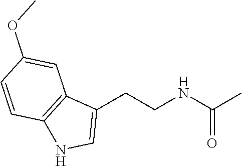 [SMILES: COc1ccc2[nH]cc(CCNC(C)=O)c2c1]

When melatonin is dispersed in water or in an oil, it settles rapidly. Minimizing the settling of the dispersed particles is a key requirement and main technical challenge in developing suspension formulations.

As used herein, the term “suspension” refers to a dispersed, two-phase system in which one phase (“internal” phase) is dispersed as solid particles in the second, liquid phase (“continuous” or “external” phase). By definition, they are thermodynamically unstable systems. Therefore, when one phase is being dispersed throughout the other phase, the system has the tendency to revert to an energetically more stable state, e.g., it undergoes aggregation, sedimentation, coalescence, crystal growth and caking.

When seeking to formulate a stable pharmaceutical suspension of a particulate active ingredient, the aim is to obtain a suspension that does not settle rapidly and/or can be easily reconstituted by gentle agitation to achieve a uniform dosage of the active ingredient. However, it is not possible to know in advance which combination of active, vehicle and excipients will give the best stability results as there is a number of factors (Brownian motion of particles, gravity, steric and electrostatic forces between particles, etc.) that have influence on the maintenance of a solid in particulate form in a stable dispersed state.

As used herein, the terms “excipient” or “additive” refer to any component, other than the active substance(s), present in a medicinal product or in a food supplement. The intended function is to act as the carrier of the active substance(s) and/or to contribute to product attributes such as efficacy, bioavailability, stability and patient acceptability and to ease the manufacturing of the product. The term “vehicle” makes reference to the carrier, composed of one or more excipients, for the active substance(s) in a liquid formulation.

As applied to a formulation of the invention, the term “% w/v” or “g/100 mL” refers to the grams of a given substance in 100 mL of the formulation, “% w/w” refers to the grams of a given substance in 100 g of the formulation and “mg/mL” means the milligrams of a given substance per mL of the formulation.

As used herein, the term “viscosity increasing agent” refers to an agent that increases the viscosity of the medium so that it minimizes inter-particle attraction and aggregation and allows the dispersed particles to settle more slowly. Exemplary viscosity increasing agents are: natural gums such as xanthan gum, tragacanth gum, guar gum; polysaccharides such as alginates, pectins, agar-agar, carrageenans, cellulose derivatives e.g. hydroxypropylmethylcellulose (HMPC), sodium carboxymethylcellulose (NaCMC), methylcellulose (MC), hydroxyethylcellulose (HEC), microcrystalline cellulose; colloidal silicon dioxide; mono-, di- and/or tri-glycerides, and mixtures thereof; and their mixtures.

As used herein, the term “preservative” relates to a compound that prevents or reduces the physical and/or chemical degradation of a composition, particularly by protecting it against microbial proliferation. Typically, preservatives according to the invention are selected from the group of methyl, ethyl and propyl parabens, benzoic acid, sodium benzoate, or their mixtures, or any other pharmaceutically acceptable antimicrobial agent.

As used herein, the term “flavouring agent” and or “sweetening agent” relates to a compound that may be added to impart a desired taste or aroma, in particular, a sweet taste. Exemplary flavouring and/or sweetening agents are natural or artificial fruit flavours selected from mint, menthol, cinnamon, vanilla, chocolate, cherry, grape, orange, strawberry or lemon.

### Formulation of the Invention

In a first aspect, the present invention relates to a liquid formulation comprising:
melatonin in particulate solid form,
a vehicle which is an oil, preferably selected from medium chain triglycerides, vegetable oils, and their mixtures; and
a viscosity increasing agent, preferably selected from: silicon dioxide, such as colloidal silicon dioxide; a composition comprising at least 50% by weight of mono-, di- or tri-glycerides or of a mixture thereof, such as a composition comprising glycerol monostearate or glyceryl dibehenate; a gum, such as xanthan gum; and their mixtures.

In a preferred embodiment, the liquid formulation is in suspension form; preferably it is in a solid-oil suspension form.

The vehicle which is an oil is comprised in the liquid formulation of the invention in an amount of at least 80% w/v; preferably, in an amount of at least 90% w/v; such as from any of these values up to 99.9% w/v, preferably up to 97% w/v, of the formulation. The upper limit of the amount of the vehicle which is an oil is 100% w/v times the density of the vehicle.

Alternatively, the vehicle which is an oil is comprised in the liquid formulation of the invention in an amount of at least 80% w/w, preferably at least 90% w/w, more preferably of at least 95% w/w, even more preferably of at least 96% w/w, such as from any of these values up to 99.9% w/w, preferably up to 99.5% w/w, of the formulation. The upper limit of the amount of the vehicle is 100% w/w minus the % w/w sum of all other components in the formulation.

In a preferred embodiment, the viscosity increasing agent is comprised in the liquid formulation of the invention in an amount of between 0.01 and 3% w/v, preferably between 0.05 and 1% w/v, more preferably between 0.1 and 0.5% w/v. In an embodiment, the liquid formulation further comprises the vehicle in the above stated amounts. Preferably, the liquid formulation is in suspension form.

Alternatively, the viscosity increasing agent is comprised in the liquid formulation of the invention in an amount of between 0.01 and 3% w/w, preferably between 0.05 and 1% w/w, more preferably between 0.1 and 0.5% w/w. In an embodiment, the liquid formulation further comprises the vehicle in the above stated amounts. Preferably, the liquid formulation is in suspension form.

In a preferred embodiment, the melatonin is in crystalline form or in an amorphous form. Preferably, it is in crystalline form.

In a particular embodiment, the melatonin in the formulation of the invention has a relative particle size distribution D90 lower than 120 μm, preferably lower than 60 μm. In a particular embodiment, the melatonin has a relative particle size distribution D90 higher than 1 μm, preferably higher than 5 μm. These maxima and minima may be combined to provide ranges.

In a particular embodiment, the melatonin has a relative particle size distribution D99 lower than 250 μm, preferably lower than 220 μm.

In a particular embodiment, the melatonin has a relative particle size distribution D10 lower than 1 μm; preferably a relative size distribution D5 lower than 1 μm. Otherwise, less than 10% of the melatonin particles are below 1 μm, particularly less than 5% are below 1 μm.

In a particular embodiment, the size of all melatonin particles in the formulation of the invention is 420 μm or lower, such as between 0.5 and 400 μm.

The size and relative particle size distribution D90 can be measured for instance by laser diffraction methods according to ISO 13320:2020, Edition 2, 2020-01.

The liquid formulations of the present invention are feasible in a wide range of melatonin concentrations, e.g. from 0.1 to 40 mg/mL, even though concentrations around 0.5 to 35 mg/ml are preferred in order to facilitate dosage and to be more adequate for paediatric patients.

In a particular embodiment, the liquid formulation of the invention comprises melatonin in an amount from about 1 to about 30 mg/mL, preferably from about 2 to about 20 mg/mL, more preferably from about 3 to about 15 mg/mL, even more preferably from about 5 to about 10 mg/mL.

Alternatively, melatonin is in an amount from about 0.05 to about 3.5% w/v, preferably from about 0.1 to about 3% w/v, more preferably from about 0.2 to about 2% w/V, even more preferably from about 0.3 to about 1.5% w/v, even more preferably from about 0.5 to about 1% w/v.

Melatonin is commercially available from a variety of chemical vendors, such as Sigma-Aldrich (Ref. M5250), Tocris Bioscience (Ref. 3550) or Nutrifoods SLU (Ref. DIE-134).

In a preferred embodiment, the vehicle, which is an oil, preferably in the above stated amounts, is medium-chain triglycerides (MCTs), also known as medium-chain triglyceride (MCT) oil. It has been found that MCTs are an advantageous vehicle as they are particularly effective at avoiding rancidity of the formulation developing upon prolonged storage. MCTs are C₆-C₁₂ fatty acid esters of glycerol. More particularly, MCTs refers to one or more C₆-C₁₂ fatty acid di- or tri-esters of glycerol. Examples of C₆-C₁₂ fatty acids are caproic acid (C₆), caprylic acid (C₈), capric acid (C₁₀), and lauric acid (C₁₂).

In a more preferred embodiment, the MCTs are present in the liquid formulation of the invention in an amount of at least 80% w/v, preferably of at least 90% w/v; such as from any of these values up to 99.9% w/v, preferably up to 97% w/v, of the formulation; such as in an amount between 90 and 99% w/v, preferably between 91 and 97% w/v, more preferably between 93 and 96% w/v of the formulation.

Alternatively, the MCTs are present in an amount of at least 80% w/w, more preferably at least 90% w/w, more preferably of at least 95% w/w, even more preferably of at least 96% w/w, such as from any of these values up to 99.9% w/w, preferably up to 99.5% w/w, more preferably up to 99.0% w/w of the formulation.

MCTs are generally obtained by the processing of coconut oil or palm oil, usually by three main stages, namely: (1) extraction of the fatty acids usually after hydrolysis or saponification, (2) their fractional distillation and (3) re esterification into triglycerides. Specific procedures are described for instance in US2015018295 A.

Alternatively, MCTs are commercially available from a variety of chemical vendors, such as Croda (Ref.: GER2395), Gustav-Hees (Ref.: 4605), Gattefose (Ref.: 3139JV1).

In another embodiment, the vehicle which is an oil, preferably in the above stated amounts, is a vegetable oil.

In a preferred embodiment, the vegetable oil is selected from corn oil, sunflower oil, safflower oil, sesame oil, soy oil, flax oil, olive oil, wheat oil, cannabis oil, peanut oil, colza oil, palm oil and their mixtures, particularly from corn oil, sunflower oil, safflower oil, sesame oil, soy oil and their mixtures. Preferably, the oil is soy oil. It has been found that soy oil is an advantageous vegetable oil as it is particularly effective at slowing rancidity of the formulation developing upon prolonged storage.

In a preferred embodiment the vegetable oil has an initial (at the time of preparing the liquid formulation of the invention) peroxide value lower than 10, preferably lower than 8, more preferably lower than 5. In a preferred embodiment the peroxide value of the vegetable oil is between 0.1 and 10, more preferably between 0.5 and 8. The peroxide value can be measured according to European Pharmacopoeia 5.0, 2.5.5. Peroxide Value.

Vegetable oils are commercially available from a variety of chemical vendors such as Croda, Gustav-Hees, Gattefosse, Altaquimica, Quimidroga, Infinity Choice.

In an embodiment, the viscosity increasing agent is selected from colloidal silicon dioxide; a composition comprising at least 70% by weight of a mixture of C₂₂ fatty acid mono- and di-esters of glycerol; a composition comprising at least 70% by weight of a mixture of C₁₆ and C₁₈ fatty acid mono-, di- and tri-esters of glycerol; and xanthan gum.

In a preferred embodiment, the viscosity increasing agent, preferably in the above stated amounts, is silicon dioxide, preferably colloidal silicon dioxide.

Colloidal silicon dioxide refers to submicron (less than a micron in diameter) silicon dioxide, and more particularly to a submicron (less than a micron in diameter) fumed silica typically prepared by vapour-phase hydrolysis of a silicon compound, such as silicon tetrachloride. The product itself is usually a light, loose, bluish-white, odourless and tasteless amorphous powder which is commercially available from a number of sources, including from DeGussa or Evonik Industries under the trade designation Aerosil™ or Cab-O-Sil™.

Preferably, the colloidal silicon dioxide exhibits a specific surface area from 175 to 225 m²/g, such as of 200 m²/g. Specific surface area can be measured according to the Brunauer, Emmett and Teller (BET) method, and more particularly according to ISO 9277:2010, Edition 2, 2010-09. Further preferably, the colloidal silicon dioxide exhibits a tamped density of 45-55 g/L, such as of 50 g/L. Tamped density can be measured according to DIN EN ISO 787/11, August 1983.

Such colloidal silicon dioxide products are known under trade name Aerosil® 200, including Aerosil® 200 F (food grade version of Aerosil® 200) and Aerosil® 200 Pharma (pharmaceutical grade version of Aerosil® 200), which are commercially available from Evonik (Ref.: 99033917).

In an embodiment, the amount of silicon dioxide, preferably colloidal silicon dioxide, in the formulation of the invention is between 0.01 and 3% w/v, preferably between 0.05 and 1% w/v, more preferably between 0.1 and 0.5% w/v, even more preferably between 0.1 and 0.3% w/v, of the formulation.

In an embodiment, the viscosity increasing agent, preferably in the above stated amounts, is a viscosity increasing composition comprising, preferably in at least 50% by weight, more preferably in at least 70% by weight, even more preferably in at least 90% by weight, such as consisting of, a mono-, di- or tri-glyceride or a mixture thereof; preferably, a mixture thereof. Preferably, the mono-, di- or triglycerides are C₁₆ to C₂₂ fatty acid esters of glycerol. These glycerides may be saturated or unsaturated. Such products can be obtained by transesterification of glycerol with triglycerides.

Said compositions are preferably present in the formulation of the invention in an amount of between 0.05 and 3% w/v, even more preferably between 0.1 and 1% w/v, of the formulation.

In a preferred embodiment, said viscosity increasing composition comprises a mono-, di- or tri-glyceride or mixture thereof which is a C₂₂ fatty acid mono-, di- or tri-ester of glycerol or a mixture thereof, preferably a C₂₂ fatty acid mono- or di-ester of glycerol or mixture thereof. More particularly, it is a behenic acid mono-, di- or tri-ester of glycerol or mixture thereof, preferably a behenic acid mono- or di-ester of glycerol or mixture thereof. Preferably, it is a mixture thereof; more preferably, it is such a mixture comprising a C₂₂ fatty acid di-ester of glycerol as majority component by weight of the mixture. More particularly, it is such a mixture comprising glyceryl dibehenate as majority component by weight of the mixture.

In any of these compositions, the combined amount by weight of the mono- and di-ester of glycerol is at least 50%, preferably at least 70%, with respect to the weight of the viscosity increasing composition.

Most preferably, the viscosity increasing agent is a composition comprising, in at least 70% by weight of the composition, a mixture of a behenic acid mono- and di-ester of glycerol, wherein glyceryl dibehenate is the majority component by weight of the mixture.

Even though comprising a mixture of glycerides, these viscosity increasing compositions are usually generally known as glyceryl dibehenate (or E471) and are available from DeGussa under the trade name Compritol®, and from Gattefose (Ref. 7101PPD) as well. A glyceryl dibehenate useful in the liquid formulations of the present invention is sold under Compritol® E ATO trademark.

In an alternative preferred embodiment, said viscosity increasing composition comprises a mono-, di- or tri-glyceride or a mixture thereof which is a C₁₆ and/or C₁₈ fatty acid mono-, di- or tri-ester of glycerol or a mixture thereof, preferably a palmitic and/or stearic acid fatty acid mono-, di- or tri-ester of glycerol or a mixture thereof. More preferably, it is a mixture of C₁₆ and/or C₁₈ fatty acid mono-, di- and tri-esters of glycerol, more particularly a mixture of palmitic and/or stearic acid mono-, di- and tri-esters of glycerol. Preferably, the mono-, di- or tri-glycerides or mixtures thereof is a mixture of C₁₆ and C₁₈ fatty acid mono-, di- and tri-esters of glycerol, more particularly a mixture of palmitic and stearic acid mono-, di- and tri-esters of glycerol.

Preferably, in such a composition, the fatty acid monoesters of glycerol are present in an amount of at least 35% w/w, such as from 35 to 60% w/w, more preferably from 42 to 52% w/w, of the composition.

Preferably, in such a composition, the fatty acid diesters of glycerol are present in an amount of at least 25% w/w, such as from 25 to 50% w/w, more preferably from 30 to 45% w/w, of the composition.

Preferably, in such a composition, the fatty acid triesters of glycerol are present in an amount of at least 1% w/w, such as from 1 to 20% w/w, more preferably from 5 to 15% w/w, of the composition.

In any of these compositions, the combined amount by weight of the mono- and di-esters of glycerol is at least 50%, preferably at least 70%, with respect to the weight of the viscosity increasing composition.

The above preferences referring to the composition comprising the mixture of C₁₆ and C₁₈ fatty acid mono-, di- and tri-esters of glycerol, more particularly the mixture of palmitic and stearic acid mono-, di- and tri-esters of glycerol, may be individually or all combined.

Even though comprising a mixture of glycerides, including those derived from palmitic acid and stearic acid, these compositions are usually known as glycerol monostearate. Such compositions are available such as from Gattefosse under Geleol® trademark (Ref.: 5749PPD).

In a further embodiment, the viscosity increasing agent, preferably in the above stated amounts, is a natural gum, more preferably xanthan gum, tragacanth gum or guar gum, even more preferably xanthan gum.

Xanthan gum (also known as E415) is a polysaccharide that can be produced from monosaccharides by a fermentation process using the bacteria Xanthomonas campestris. It is commercially available from a variety of chemical vendors.

A preferred xanthan gum is a fine particle size xanthan gum. “Fine particle size” herein means that at least 90%, and preferably all, of xanthan gum particles are sized not greater than 180 μm. Particle size can be measured by shaking 50 g product on a 80 mesh (180 μm) Tyler Standard Screen for 20 minutes using a Ro-Tap sieve shaker. Such products are commercially available from Azelis under Xantural® 75 trademark (Ref.: BP106877) or from CPKelkco under the same trade name (Ref 454-X).

In an embodiment, the amount of xanthan gum in the formulation of the invention is between 0.01 and 1% w/v, even more preferably between 0.05 and 0.2% w/v, of the formulation.

The inventors have importantly found that the combination of the vehicle which is an oil with the suspending agent results in an improved physical stability of the suspension of melatonin without worsening other features such as chemical stability, organoleptic properties and/or microbial activity.

In a particular embodiment, the liquid formulation, preferably a suspension, of the invention may further comprise a preservative, selected from the group of methyl, ethyl and propyl parabens, benzoic acid, sodium benzoate, or their mixtures, or any other pharmaceutically acceptable antimicrobial agent.

However, surprisingly, there is no need to add a preservative to avoid the microbial contamination of the liquid formulation of the present invention. Therefore, in a particularly preferred embodiment, the liquid formulations of the present invention do not comprise a preservative selected from any one or more, such as all, of those described above. More preferably, the liquid formulations of the present invention do not comprise a preservative.

In a particular embodiment, the liquid formulation, preferably a suspension, of the invention may further comprise a sweetening agent and/or a flavouring agent.

However, surprisingly, there is no need to add neither a sweetening agent nor a flavouring agent to obtain an organoleptically pleasant liquid formulation. Therefore, in a particularly preferred embodiment, the liquid formulations of the present invention do not comprise neither a sweetening agent nor a flavouring agent.

The liquid formulation of the invention may include other excipients. However, the inventors have found that the formulations of the present invention have excellent properties even in the absence of additional excipients. Therefore, in an embodiment, the liquid formulation does not comprise further excipients in addition to the vehicle and the viscosity increasing agent.

In a particular embodiment, the formulations of the invention do not comprise a surfactant selected from polyoxyethanyl-tocopheryl-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof. In an embodiment, the formulations of the invention do not comprise a surfactant in addition to the vehicle and/or the viscosity increasing agent.

In a particular embodiment, the formulations of the invention do not include a gelling agent in addition to the vehicle and/or the viscosity increasing agent and are not in the form of a gel.

In a particular embodiment, the formulations of the invention do not comprise an acrylic polymer, more particularly they do not comprise a polymer.

In an embodiment, the formulations of the invention comprise melatonin and optionally only one further active ingredient. Preferably, the formulations of the invention comprise melatonin as sole active ingredient.

In an embodiment, the formulations of the invention do not comprise tetrahydro-cannabinol; preferably do not comprise a cannabinoid; more preferably do not comprise a cannabinoid, cannabinoid extract (i.e. an extract from a cannabis plant, the extract comprising a cannabinoid), terpene, and/or terpene extract (i.e. an extract from a plant, the extract comprising a terpene); even more preferably do not comprise a cannabinoid, cannabinoid extract, terpene, terpene extract, ethyl pyruvate, caffeine and/or resveratrol.

In an embodiment, the formulations of the invention do not comprise insulin.

In an embodiment, the formulations of the invention do not comprise unsubstituted or substituted diindolylmethane, preferably they do not comprise a compound comprising two or more unsubstituted or substituted indole groups.

In an embodiment, the formulations of the invention do not comprise pyrroloquinoline quinone, preferably they do not comprise pyrroloquinoline quinone nor a derivative thereof, more preferably they do not comprise pyrroloquinoline quinone nor a derivative thereof of the following formula:

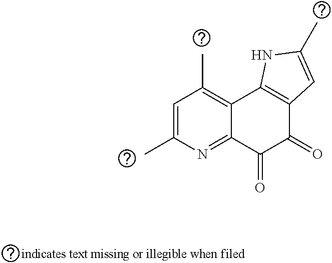

wherein R₁, R₂, and R₃ are, independently, a hydrogen atom, an alkyl group, an alkenyl group, a haloalkyl group, a benzyl group, or an alkoxycarbonylalkyl group; nor a salt thereof.

In an embodiment, the formulations of the invention do not comprise Cyclosporine A, preferably they do not comprise any peptide.

In an embodiment, the formulations of the invention do not comprise a chitosan salt salified with N-acetylcysteine (NAC), preferably they do not comprise chitosan or any salt thereof.

In a more particular embodiment, the liquid formulation consists of:
melatonin in particulate solid form,
the vehicle as described in any of the above embodiments; and
the viscosity increasing agent as described in any of the above embodiments.

In an embodiment, the formulation of the present invention comprises, or in particular consists of:
melatonin in particulate solid form, preferably in an amount from 1 to 30 mg/mL, preferably from 2 to 20 mg/mL, more preferably from 3 to 15 mg/mL, even more preferably from 5 to 10 mg/mL, of the formulation,
a vehicle which is an oil, wherein the oil is preferably selected from medium chain triglycerides, vegetable oils and their mixtures, and is preferably present in an amount of at least 80% w/v, preferably of at least 90% w/v, such as from any of these values up to 99.9% w/v, preferably up to 97% w/v, of the formulation, and
a viscosity increasing agent, preferably selected from: silicon dioxide, preferably colloidal silicon dioxide; a composition comprising at least 70% by weight of a mixture of mono-, di- or tri-glycerides, wherein said mono-, di- or tri-glycerides are selected from C₁₆ to C₂₂ fatty acid esters of glycerol; a gum, such as xanthan gum; and their mixtures; preferably in an amount between 0.01 and 3% w/v, more preferably between 0.05 and 1% w/v, even more preferably between 0.1 and 0.5% w/v, of the formulation.

In an embodiment, the formulation of the present invention comprises, or in particular consists of:
melatonin in particulate solid form, in an amount from 1 to 30 mg/mL, preferably from 2 to 20 mg/mL, more preferably from 3 to 15 mg/mL, even more preferably from 5 to 10 mg/mL, of the formulation,
a vehicle which is medium chain triglycerides, in an amount of at least 80% w/v, preferably of at least 90% w/v, more preferably between 90 and 99% w/v, even more preferably between 91 and 97% w/v, yet more preferably between 93 and 96% w/v of the formulation, and
a viscosity increasing agent, preferably selected from: silicon dioxide, preferably colloidal silicon dioxide; a composition comprising at least 70% by weight of a mixture of mono-, di- or tri-glycerides, wherein said mono-, di- or tri-glycerides are selected from C₁₆ to C₂₂ fatty acid esters of glycerol; a gum, such as xanthan gum; and their mixtures; preferably in an amount between 0.01 and 3% w/v, more preferably between 0.05 and 1% w/v, even more preferably between 0.1 and 0.5% w/v, of the formulation.

In another embodiment, the formulation of the present invention comprises, or in particular consists of:
melatonin in particulate solid form, preferably in an amount from 1 to 30 mg/mL, preferably from 2 to 20 mg/mL, more preferably from 3 to 15 mg/mL, even more preferably from 5 to 10 mg/mL, of the formulation,
a vehicle which is an oil, wherein the oil is preferably selected from medium chain triglycerides, vegetable oils and their mixtures, and is preferably present in an amount of at least 80% w/v, preferably of at least 90% w/v, such as from any of these values up to 99.9% w/v, preferably up to 97% w/v, of the formulation, and
a viscosity increasing agent which is colloidal silicon dioxide, preferably in an amount between 0.01 and 3% w/v, more preferably between 0.05 and 1% w/v, even more preferably between 0.1 and 0.5% w/v, yet more preferably between 0.1 and 0.3% w/v, of the formulation.

In an embodiment, the formulation of the present invention comprises, or in particular consists of:
melatonin in particulate solid form, in an amount from 1 to 30 mg/mL, preferably from 2 to 20 mg/mL, more preferably from 3 to 15 mg/mL, even more preferably from 5 to 10 mg/mL, of the formulation,
a vehicle which is medium chain triglycerides, in an amount of at least 80% w/V, preferably of at least 90% w/v, more preferably between 90 and 99% w/v, even more preferably between 91 and 97% w/v, yet more preferably between 93 and 96% w/v, of the formulation, and
a viscosity increasing agent which is colloidal silicon dioxide, preferably in an amount between 0.01 and 3% w/v, preferably between 0.05 and 1% w/v, more preferably between 0.1 and 0.5% w/v, even more preferably between 0.1 and 0.3% w/v, of the formulation.

In a preferred embodiment, the formulation of the present invention comprises, or in particular consists of:
melatonin in a particulate solid form, in an amount from 2 to 20 mg/mL, preferably from 3 to 15 mg/mL, more preferably from 5 to 10 mg/mL, of the formulation,
a vehicle which is medium chain triglycerides in an amount between 90 and 99% w/v, preferably between 91 and 97% w/v, more preferably between 93 and 96% w/v, of the formulation, and
a viscosity increasing agent, which is colloidal silicon dioxide in an amount between 0.1 and 0.5% w/v, preferably between 0.1 and 0.3% w/v, of the formulation.

In a preferred embodiment, the formulation of the present invention comprises, or in particular consists of:
melatonin in a particulate solid form in an amount from 3 to 15 mg/mL, preferably from 5 to 10 mg/ml of the formulation,
medium chain triglycerides in an amount between 91 and 97% w/v of the formulation,
colloidal silicon dioxide in an amount between 0.1 and 0.3% w/v of the formulation.

The formulations of the invention have a relative density between 0.9 and 1 g/mL, preferably between 0.93 and 0.96 mg/mL. Relative density can be measured by pycnometer or hydrometer (20° C.). More specifically, relative density can be measured according to European Pharmacopoeia 5.0, 2.2.5. Relative Density.

### Methods

The formulations of the invention can be prepared following any known process of the prior art.

In a second aspect, the present invention is directed to a method for preparing the formulation of the invention, wherein said method comprises the following steps:
a) Mixing the vehicle in liquid form with the viscosity increasing agent in solid form;
b) Adding the melatonin in particulate solid form into the mixture obtained in step a) and mixing.

Thus, the present invention is directed to a method for preparing the formulation of the invention, wherein said method does not comprise, prior to step a) or b), a step for preparing a melatonin composition comprising melatonin and an excipient, wherein the melatonin composition is in solid form, particularly wherein the melatonin composition is a powder obtained by a freeze-drying process.

Particularly, the method does not comprise, prior to step a) or b), a step for preparing a melatonin composition comprising melatonin and a suspending agent, particularly wherein the melatonin composition is a powder obtained by a freeze-drying process. More particularly, said suspending agent is hydrolyzed beeswax, even more particularly wherein the hydrolyzed beeswax is obtained from natural beeswax through saponification treatment and acidification treatment with at least one food-grade organic acid in sequence.

In a preferred embodiment, the mixing in step a) is performed in a suitable tank equipped with a stirrer until a homogeneous mix is obtained.

Optionally, the method comprises adding a sweetening agent and/or a flavouring agent and/or a preservative in any of the above steps. Preferably, the method does not comprise adding neither a sweetening nor a flavouring agent, more preferably the method does not comprise adding neither a sweetening nor a flavouring agent nor a preservative.

In a preferred embodiment, the method further comprises a step c) of adding further vehicle in liquid form (preferably q.s. to final volume) and mixing. Preferably, the vehicle in liquid form is the same vehicle in liquid form as in step a).

Particularly, the present invention is directed to a method for preparing the formulation of the invention wherein said method consists of the following steps:
a) Mixing the vehicle in liquid form with the viscosity increasing agent in solid form;
b) Adding the melatonin in particulate solid form into the mixture obtained in step a) and mixing;
c) Adding the vehicle in liquid form up to final volume and mixing.

In a preferred embodiment, the method comprises a final step of homogenising the obtained mixture.

Particularly, the present invention is directed to a method for preparing the formulation of the invention wherein said method consists of the following steps:
a) Mixing the vehicle in liquid form with the viscosity increasing agent in solid form;
b) Adding the melatonin in particulate solid form into the mixture obtained in step a) and mixing;
c) Adding the vehicle in liquid form up to final volume and mixing.
d) Homogenising the mixture obtained in step c).

As used herein, the term “homogenous” means that the final mixture has substantially the same composition throughout. Particularly, the mixture is considered homogeneous when samples of the formulation from various areas in a container containing it, such as from the top, middle, and bottom, reveal a concentration of melatonin differing in at most 10%.

Meanings and preferences for each component of the formulation are those described for the formulation in the first aspect of the invention. In a preferred embodiment, the viscosity increasing agent is colloidal silicon dioxide and the vehicle is medium chain triglycerides or a mixture of medium chain triglycerides.

In a different aspect, the invention refers to a formulation obtainable by any of the methods described herein.

### Uses

In a further aspect, the present invention relates to a formulation of the invention as defined in any of the embodiments described above, for use as a medicament and/or a food supplement.

Another aspect of the invention is directed to a liquid formulation of the invention as defined in any of the embodiments described above, for use in the prevention or treatment of sleep disorders such as insomnia and circadian rhythm sleep-wake disorders.

The term “treatment” or “to treat” in the context of this specification means administration of a formulation according to the invention to ameliorate or eliminate a condition or disorder or one or more symptoms associated with said condition or disorder. “Treatment” also encompasses ameliorating or eliminating the physiological sequelae of the condition.

The term “prevention” or “to prevent” in the context of this specification means reducing the risk of acquiring or developing the condition or disorder or one or more symptoms associated with said condition or disorder.

Circadian rhythm sleep disorders—formally known as circadian rhythm sleep-wake disorders—are a group of conditions tied to dysfunctions or misalignments with the body's internal clock. Examples of these disorders include mild conditions such as jet lag, as well as more debilitating conditions such as delayed and advanced sleep-wake disorder, irregular sleep-wake rhythm disorder and shift work disorder.

The above aspect can be formulated as the use of a formulation of the invention as defined in any of the embodiments described above, in the manufacture of a medicament and/or a food supplement for the prevention or treatment of sleep disorders such as insomnia and circadian rhythm sleep-wake disorders.

The above aspect can be formulated as the use of a formulation of the invention, as defined in any of the embodiments described above, as a medicament and/or a food supplement for the prevention or treatment of sleep disorders such as insomnia and circadian rhythm sleep-wake disorders.

The above aspect can be formulated as a method of treating or preventing sleep disorders such as insomnia and circadian rhythm sleep-wake disorders, the method comprising the administration of a formulation of the invention as defined in any of the embodiments described above to a patient in need of such prevention or treatment.

Preferably, in any of the above embodiments, the formulations of the invention are suitable for oral use, more particularly for oral use by ingestion. More particularly, in any of the above medical use embodiments, the formulation of the invention is administered by the oral route, more particularly by ingestion.

In an embodiment, in any of the above medical use embodiments, the formulation of the invention is administered to a non-adult, more particularly to a subject aged under 3 years old.

The following examples are merely illustrative of certain embodiments of the invention and cannot be considered as restricting it in any way.

### Example 1

A formulation according to the invention was prepared by first transferring 304.5 kg of medium chain triglycerides (in liquid form) to a suitable tank. Afterwards, adding 0.6 kg of silicon dioxide (in solid form) to the tank and keeping under stirring at 2900 rpm for 15 minutes, and then, adding 4.0 kg of melatonin (in particulate solid form) to the tank under stirring at 2900 rpm. Finally, adding 76.1 kg of medium chain triglycerides (in liquid form) to the tank and keeping under stirring at 2900 rpm for 45 minutes.

### Example 2. Melatonin Oral Suspension

| Ingredient | Quantity (g/100 mL-% w/v) |
|---|---|
| Melatonin | 0.90 |
| Silicon dioxide colloidal | 0.15 |
| Medium chain triglycerides | q.s. to 100 ml |

### Example 3. Melatonin Oral Suspension

| Ingredient | Quantity (g/100 mL-% w/v) |
|---|---|
| Melatonin | 0.60 |
| Silicon dioxide colloidal | 0.15 |
| Medium chain triglycerides | q.s. to 100 ml |

### Example 4. Melatonin Oral Suspension

| Ingredient | Quantity (g/100 mL-% w/v) |
|---|---|
| Melatonin | 0.90 |
| Glycerol monostearate (Geleol ®) | 0.50 |
| Medium chain triglycerides | q.s. to 100 ml |

### Example 5. Melatonin Oral Suspension

| Ingredient | Quantity (g/100 mL-% w/v) |
|---|---|
| Melatonin | 0.90 |
| Xanthan gum (Xantural ® 75) | 0.12 |
| Medium chain triglycerides | q.s. to 100 ml |

### Example 6. Melatonin Oral Suspension

| Ingredient | Quantity (g/100 mL-% w/v) |
|---|---|
| Melatonin | 0.90 |
| Glyceryl dibehenate (Compritol ® E ATO) | 0.50 |
| Soybean oil | q.s. to 100 ml |

### Example 7. Stability Studies

For the purposes of the present invention, the stability of two oily suspensions of melatonin in different containers was tested.

The suspensions were prepared as reported above, filled into the packaging materials detailed below and stored under the conditions mentioned below, in order to determine if the tested multiple-dose suspensions remain stable during 6 months:

| Melatonin | | Storage |
|---|---|---|
| suspensions | Pre-stability packaging | Conditions |
| L21/05 | MA-360A: Topaz glass | 25° C./60% RH |
| (6 mg/mL) | bottle 30 ml Eladiet | |
| L21/06 | MA-361A: White cap diameter | 40° C./75% RH |
| (9 mg/mL) | 10 PE-HD WEI3 Eladiet | |
| Melatonin | | Storage |
| suspensions | In use pre-stability packaging | Conditions |
| L21/05 | MA-360A: Topaz glass bottle | 5° C. |
| (6 mg/mL) | 30 mL Eladiet | |
| L21/06 | MA-362A: 1 mL glass | 25° C./60% |
| (9 mg/mL) | pipette with dropper | RRH |
| | DIN 168 GL-18 Eladiet | 40° C./75% RH |
| Melatonin | Pre-stability | Storage |
| suspensions | packaging | Conditions |
| L21/05 | MA-293A: Topaz glass bottle | 25° C./60% RH |
| (6 mg/mL) | 30 mL GL18 SR Special | |
| L21/06 | MA-286A: White child-proof | 40° C./75% RH |
| (9 mg/mL) | dropper cap | |

L21/05 is the formulation of Example 3 and L21/06 the formulation of Example 2.

After 6 months of storage, the physicochemical stability of the suspensions was verified by checking appearance, organoleptic characteristics and redispersion capability, and measuring density (Ph Eur. 2.2.5.) and melatonin concentration (HPLC/UV spectroscopy).

Packaging: 30 ml ambar glass bottle with white cap diameter 10 PE-HD WEI3
t = 6 months

| | | | | | 40° C./75% |
|---|---|---|---|---|---|
| | | 25° C./60% RH | 40° C./75% RH | 25° C./60% RH | RH |
| | Theorical Values | (L 21/05) | (L 21/05) | (L 21/06) | (L 21/06) |
| Appearance and | Homogeneous off-white | Complies | Complies | Complies | Complies |
| organoleptic | oily suspension | | | | |
| characteristics | | | | | |
| Redispersion | Particles | Complies | Complies | Complies | Complies |
| | homogeneously | | | | |
| | suspended | | | | |
| Relative density | 0.93-0.96 g/ml | 0.95 | 0.95 | 0.95 | 0.95 |
| Melatonin | 5.4-6.6 mg/ml (L 21/05) | 5.8 | 6.2 | 8.8 | 9.0 |
| concentration | 8.1-9.9 mg/ml (L 21/06) | (97%) | (103%) | (98%) | (100%) |

Packaging: 30 ml amber glass bottle with 1 ml glass pipette with dropper DIN 168 GL-18
t = 6 months

| | | | 25° C./ | 40° C./ | | 25° C./ | 40° C./ |
|---|---|---|---|---|---|---|---|
| | | 5° C. | 60% RH | 75% RH | 5° C. | 60% RH | 75% RH |
| | Theorical Values | (L21/05) | (L21/05) | (L21/05) | (L21/06) | (L21/06) | (L21/06) |
| Appearance | Homogeneous | Complies | Complies | Complies | Complies | Complies | Complies |
| and | off-white oily | | | | | | |
| organoleptic | suspension | | | | | | |
| characteristics | | | | | | | |
| Redispersion | Particles | Complies | Complies | Complies | Complies | Complies | Complies |
| | homogeneously | | | | | | |
| | suspended | | | | | | |
| Relative | 0.93-0.96 g/ml | 0.95 | 0.95 | 0.95 | 0.95 | 0.95 | 0.95 |
| density | | | | | | | |
| Melatonin | 5.4-6.6 mg/ml | 5.7 | 5.8 | 5.5 | 9.0 | 8.9 | 8.9 |
| concentration | (L21/05) | (95%) | (96%) | (93%) | (100%) | (99%) | (99%) |
| | 8.1-9.9 mg/ml | | | | | | |
| | (L21/06) | | | | | | |

Packaging: 30 mL amber glass bottle GL18 SR with white child-proof dropper cap
t = 6 months

| | | | | | 40° C./75% |
|---|---|---|---|---|---|
| | | 25° C./60% RH | 40° C./75% RH | 25° C./60% RH | RH |
| | Theorical Values | (L21/05) | (L21/05) | (L21/06) | (L21/06) |
| Appearance | Homogeneous off-white | Complies | Complies | Complies | Complies |
| and | oily suspension | | | | |
| organoleptic | | | | | |
| characteristics | | | | | |
| Redispersion | Particles homogeneously | Complies | Complies | Complies | Complies |
| | suspended | | | | |
| Relative | 0.93-0.96 g/ml | 0.95 | 0.95 | 0.95 | 0.95 |
| density | | | | | |
| Melatonin | 5.4-6.6 mg/ml (L21/05) | 5.6 | 6.0 | 8.9 | 8.9 |
| concentration | 8.1-9.9 mg/ml (L21/06) | (94%) | (99%) | (99%) | (98%) |

According to the results, the liquid formulations of the present invention remain stable under all the studied conditions for at least 6 months.

### Example 8. Further Compositions

Further melatonin oral suspensions according to the invention were manufactured. In these further suspensions, melatonin is present in a concentration of 9 mg/mL. Each further suspension refers to a combination of a specific oily vehicle and a specific viscosity increasing agent, as follows (percentages in % w/v i.e. g/100 mL):

L22/05: MCTs + Glycerol monostearate (Geleol ®) 0.5%
L22/06: MCTs + Glyceryl dibehenate (Compritol ® E ATO) 0.5%
L22/07: MCTs + Xanthan gum (Xantural ® 75) 0.12%
L22/09: Corn Oil + Glycerol monostearate (Geleol ®) 0.5%
L22/10: Corn Oil + Glyceryl dibehenate (Compritol ® E ATO) 0.5%
L22/11: Corn Oil + Xanthan gum (Xantural ® 75) 0.12%
L22/13: Safflower Oil + Glycerol monostearate (Geleol ®) 0.5%
L22/14: Safflower Oil + Glyceryl dibehenate (Compritol ® E ATO) 0.5%
L22/15: Safflower Oil + Xanthan gum (Xantural ® 75) 0.12%
L22/17: Sunflower Oil + Glycerol monostearate (Geleol ®) 0.5%
L22/18: Sunflower Oil + Glyceryl dibehenate (Compritol ® E ATO) 0.5%
L22/19: Sunflower Oil + Xanthan gum (Xantural ® 75) 0.12%
L22/21: Soy Oil + Glycerol monostearate (Geleol ®) 0.5%
L22/22: Soy Oil + Glyceryl dibehenate (Compritol ® E ATO) 0.5%
L22/23: Soy Oil + Xanthan gum (Xantural ® 75) 0.12%
L22/25: Sesame Oil + Glycerol monostearate (Geleol ®) 0.5%
L22/26: Sesame Oil + Glyceryl dibehenate (Compritol ® E ATO) 0.5%
L22/27: Sesame Oil + Xanthan gum (Xantural ® 75) 0.12%
L22/29: MCTs + Aerosil ® 200 F 0.16%
L22/30: Corn Oil + Aerosil ® 200 F 0.16%
L22/31: Safflower Oil + Aerosil ® 200 F 0.16%
L22/32: Sunflower Oil + Aerosil ® 200 F 0.16%
L22/33: Soy Oil + Aerosil ® 200 F 0.16%
L22/34: Sesame Oil + Aerosil ® 200 F 0.16%

### Example 9. Stability Testing for Further Compositions

The compositions of Example 8 were subjected to stability studies as in Example 7.

Pre-packaging was in Topaz glass bottles. The compositions were tested after 3, 6 and 12 months of storage, both at 25° C./60% RH and 40° C./75% RH.

Each composition, at every time point and condition tested, was in the form of a clear oily suspension with visible solid particles in suspension and/or precipitates.

Each composition, at every time point and condition tested, showed visible solid particles homogeneously suspended, with no precipitates, following redispersion.

Each composition, at every time point and condition tested, showed adequate density values within 0.93-0.96 g/mL.

Each composition, at every time point and condition tested, showed a melatonin concentration within 7.2-10.8 mg/ml.

According to these results, it can be concluded that the liquid formulations of the present invention remain stable under all the studied conditions for at least 6 months.

1. A liquid formulation comprising:
melatonin in particulate solid form,
a vehicle which is an oil, comprised in the liquid formulation in an amount of at least 80% w/v,
a viscosity increasing agent.

2. The formulation according to claim 1, wherein the formulation is in the form of a suspension.

3. The formulation according to claim 1, wherein the amount of melatonin is from 1 to 30 mg/mL.

4. The formulation according to claim 1, wherein the vehicle which is an oil is selected from medium chain triglycerides and vegetable oils.

5. The formulation according to claim 4, wherein the vehicle which is an oil is medium chain triglycerides.

6. The formulation according to claim 1, wherein the amount of oil comprised in the liquid formulation is expressed as at least 90% w/w.

7. The formulation according to claim 1, wherein the viscosity increasing agent is selected from: colloidal silicon dioxide; a composition comprising at least 70% by weight of a mixture of mono-, di- and/or tri-glycerides, wherein said mono-, di- or tri-glycerides are selected from C₁₆ to C₂₂ fatty acid esters of glycerol; and xanthan gum.

8. The formulation according to claim 1, wherein the viscosity increasing agent is colloidal silicon dioxide, and is comprised in the formulation in an amount of between 0.01 and 3% w/v of the formulation.

9. The formulation according to claim 7, wherein the viscosity increasing agent is a composition comprising at least 70% by weight of a mixture of mono-, di- and/or tri-glycerides, wherein said mono-, di- or tri-glycerides are selected from C₁₆ to C₂₂ fatty acid esters of glycerol, and wherein said composition is present in the formulation in an amount of between 0.05 and 3% w/v of the formulation.

10. The formulation according to claim 9, wherein the viscosity increasing agent is a composition comprising at least 70% by weight of a mixture of mono- and di-glycerides, wherein said mono- and di-glycerides are C₂₂ fatty acid esters of glycerol.

11. The formulation according to claim 9, wherein the viscosity increasing agent is a composition comprising at least 70% by weight of a mixture of mono-, di- and tri-glycerides, wherein said mono-, di- and tri-glycerides are C₁₆ and/or C₁₈ fatty acid esters of glycerol.

12. The formulation according to claim 1, comprising:
melatonin in particulate solid form, in an amount from 2 to 20 mg/mL,
medium chain triglycerides in an amount between 90 and 99% w/v,
colloidal silicon dioxide in an amount between 0.1 and 0.5% w/v.

13. The formulation according to claim 1, in a form suitable for oral administration by ingestion.

14. A method for preparing a formulation as defined in claim 1, said method comprising the following steps:
mixing the vehicle which is an oil in liquid form with the viscosity increasing agent in solid form; and
adding the melatonin in particulate form into the mixture obtained in step a) and mixing.

15. A method of treating or preventing insomnia and circadian rhythm sleep-wake disorders which comprises administering a therapeutically effective amount of a formulation as defined in claim 1 to a patient in need thereof.

16. The method according to claim 15, wherein the formulation is administered orally.

17. The formulation according to claim 3, wherein the amount of melatonin is from 2 to 20 mg/mL.

18. The formulation according to claim 8, wherein the viscosity increasing agent is colloidal silicon dioxide, and is comprised in the formulation in an amount of between 0.05 and 1% w/v of the formulation.

19. The formulation according to claim 9, wherein said composition is present in the formulation in an amount of between 0.1 and 1% w/v of the formulation.

20. The formulation according to claim 12, comprising:
melatonin in particulate solid form, in an amount from 3 to 15 mg/mL,
medium chain triglycerides in an amount between 91 and 97% w/v,
colloidal silicon dioxide in an amount between 0.1 and 0.3% w/v.
